(19) United States
(12) Patent Application Publication
(10) Pub. No.: US 2003/0092092 A1
(43) Pub. Date: May 15, 2003

(54) Antitryptase autoantibody-tryptase immune complex as a unique marker for diagnosis and as a potential target for therapy of immediate hypersensitivity diseases

(75) Inventors: William M. Pandak, Richmond, VA (US); Hae Ki Min, Richmond, VA (US); Shunlin Ren, Richmond, VA (US)

Correspondence Address: DAVID A. HALL, HELLER EHRMAN ET AL., 4350 LA JOLLA VILLAGE DRIVE #700, SAN DIEGO, CA, 92122, US

(73) Assignee: PAX BIOGEN CO., LTD.

(21) Appl. No.: 10/060,545

(22) Filed: Jan. 29, 2002

(30) Foreign Application Priority Data
Nov. 14, 2001 (KR) 2001-70732

(51) Int. Cl. G01N 33/53; G01N 33/537; G01N 33/543

(52) U.S. Cl. 435/7.93

(57) ABSTRACT

The disclosure describes the presence of antitryptase autoantibody-tryptase immune complexes (the immune complexes) in sera of patients with immediate hypersensitivity diseases. Specifically, the present invention relates to a method for diagnosing of the diseases by detecting of the immune complex. More specifically the present invention includes the discovery of antitryptase autoantibody-tryptase immune complex in anaphylaxis sera and establishment of enzyme linked immunosorbent assays (ELISA) for detection of the antitryptase autoantibody-mast cell tryptase immune complex in sera of patients with anaphylaxis. The immune complexes can be used as a target for therapy of the immediate hypersensitivity diseases. The pathophysiological mechanism is provided.

### RELATED APPLICATIONS

Benefit of priority is claimed under 35 U.S.C. §119(a) to KR 2001-70732, filed Nov. 14, 2001, is claimed herein. The subject matter of this application is incorporated herein in its entirety.

### TECHNICAL FIELD

The present invention relates to a method for diagnosing the diseases by detecting antitryptase autoantibody-mast cell tryptase immune complexes. Alos provided is a kit for performing the assay. More specifically the present invention includes the discovery of autoantibody-mast cell tryptase immune complex in anaphylaxis sera and establishment of enzyme linked immunosorbent assays (ELISA) for detection of autoantibody-mast cell tryptase immune complexes in sera of patients with anaphylaxis. The presence of the immune complexes be potentially used as a target for the therapy of the immediate hypersensitivity diseases.

BIBLIOGRAPHY

Complete bibliographic citations to the non-patent references cited herein can be found in the Bibliography section, immediately preceding the claims.

### BACKGROUND

Anaphylaxis. Anaphylaxis is a clinical manifestation of immediate hypersensitivity that occurs after the interaction between a specific antigen and a homocytotrophic antibody. This occurs rapidly, often is dramatic, and is unanticipated. Death may occur through airway obstruction or irreversible vascular collapse. Recent data shows that of the 1999 US population of 272 million including our veterans, the number of people at risk for anaphylaxis is up to 40.9 million in the USA each year (1). The etiology and pathophysiology of immediate hypersensitivity anaphylaxis are obscure. Classic anaphylaxis occurs when an allergen combines with IgE antibody bound to the surface membranes of mast cells and circulating basophils. The interaction between IgE-antigen and the membranes activate a complex series of events, resulting in the release of many mediators of inflammation, including histamine, tryptases, prostaglandins, and leukotrienes. However, no evidence shows that these mediators account for all of the observed physiological changes seen in anaphylaxis.

Human mast cell tryptase. The tryptase released from mast cells appears critical for mediating anaphylaxis (2). Tryptase (EC3.4.21.59) is the most abundant neutral protease and protein component in human mast cell secretory granules. It is secreted from the activated mast cells in parallel with histamine (2), and ionically bound to heparin (3). In vitro, tryptase cleaves and inactivates fibrinogen as a coagulable substrate for thrombin (4), degrades fibronectin (5) as well as other neuropeptides (5-8). Enhanced contractility of bronchial smooth muscle to histamine (5;9), proliferation of fibroblasts (5; 10), bronchial epithelial cells (11) and tracheal smooth muscle cells (12; 13), stimulation of collagen synthesis by fibroblasts (5;14), and recruitment of eosinophils (15) are other in vitro activities of human tryptase. Although, several of these inflammatory mediators appear to play a role in the development of an anaphylactic reaction, the mechanism remains unclear, suggesting the involvement of other yet to be identified mediators or mechanism (2). Furthermore, the presentation/generation of the known inflammatory mediators in response to allergens appear predictable, while the development/progression to anaphylaxis is currently unpredictable. The reason for this is unclear.

### SUMMARY OF THE INVENTION

The goal of the present invention is to provide an immunoassay for detecting of antitryptase autoantibody-mast cell tryptase complex. Provided is an assay that is extremely sensitive, accurate, and precise, and does not require the use of radioactive isotopes. A new mechanism supported by the data that provides better understanding of immediate hypersensitivity diseases is provided.

The present invention relates to a method for diagnosing the diseases by using autoantibody-mast cell tryptase immune complex. More specifically the present invention includes the discovery of autoantibody-mast cell tryptase immune complex in anaphylaxis serum and establishment of enzyme linked immunosorbent assays (ELISA) for detection of autoantibody-mast cell tryptase immune complex in patient sera with anaphylaxis. Kits for performing the assays are provided.

The present assay is a double antibody-sandwich enzyme-linked immunosorbent assay (ELISA) for the detection of human autoantibody-mast cell tryptase immune complex in patient serum. The ELISA of the present invention comprises capture antibodies and detection antibodies. The capture antibodies are monoclonal antibodies tryptase specific antibodies capable of capturing human tryptase from human serum and the detection antibodies are monoclonal antibodies human IgG specific capable to detect IgG in the complex.

The present disclosure for the first time to demonstrates that 1. Anaphylaxis could be an autoimmune disease; 2. Presence of autoantibody-mast cell tryptase immune complex in anaphylaxis serum; 3. Provides a new method to detect the autoantibody-mast cell tryptase immune complex in patient serum, which does not require the use of radioactive reagents which are inherently dangerous; 4. The autoantibody-mast cell tryptase can be a potential target for immunotherapy of anaphylaxis diseases.

### BRIEF DESCRIPTION OF THE DRAWINGS

FIG. 1 shows the roles of human mast cell tryptase in complement and anaphylatoxin metabolism.

FIG. 2 shows Diagram of ELISA assay for detecting antitryptase autoantibody-tryptase immune complex in sera of patients with immediate hypersensitivity diseases. Anti-mast cell tryptase antibody was coated in 96 well as capture and the immune complexes in sera of patients were detected by alkaline phosphate conjugated anti human Ig Fc Ab.

FIG. 3a shows titers of antitryptase autoantibody-tryptase immune complexes in sera of patients with anaphylaxis (N=30) against normal subjects (N=16) detected by ELISA assay using antitryptase antibodies as capture. Serum was used to measure the complex in all subjects. Each value represents mean of two or more determinations. FIG. 3b shows the correlation of the immune complexes with β-tryptase levels.

FIG. 4 is dose course of the sandwich ELISA for antitryptase autoantibody-tryptase immune complex. Sample 1 and 2 are patients with anaphylaxis.

FIG. 5 shows that the purified human mast cell tryptase competes the immune complex binding in ELISA assay. The antitryptase antibody coated 96well plate incubated with antitryptase autoantibody positive sera with or without the purified mast cell tryptase(0-150 nM) for 2 hrs at room temperature. The bound human IgG was then detected by alkaline phosphotase conjugated anti-human IgG Fc specific antibody.

FIG. 6 shows the effect of Rheumatoid Factor (RF) on Antitryptase autoantibody-tryptase immune complex ELISA Assay.

### DETAILED DESCRIPTION OF THE PREFERRED EMBODIMENTS

Tryptase may play an important role in anaphylatoxin metabolism.

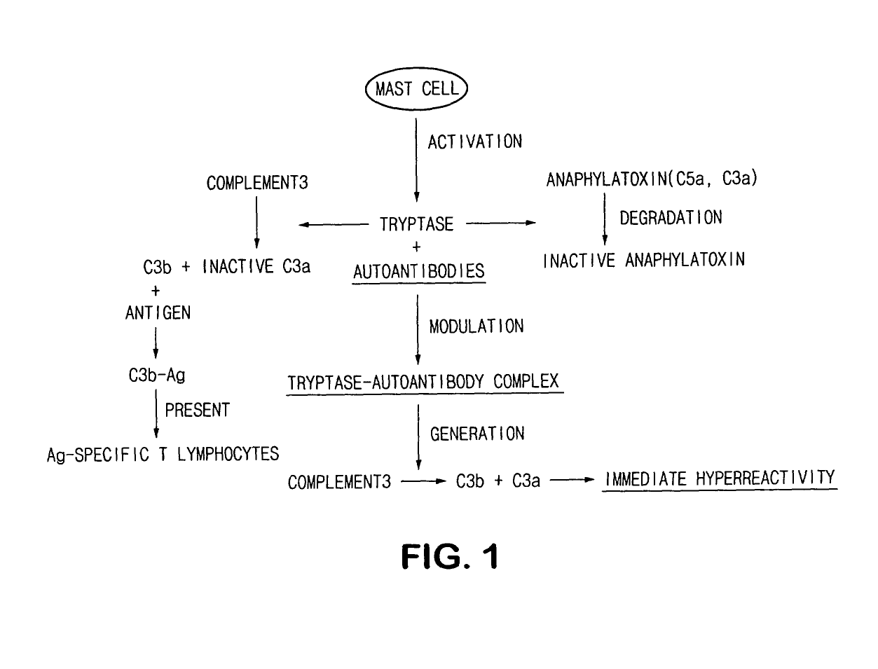

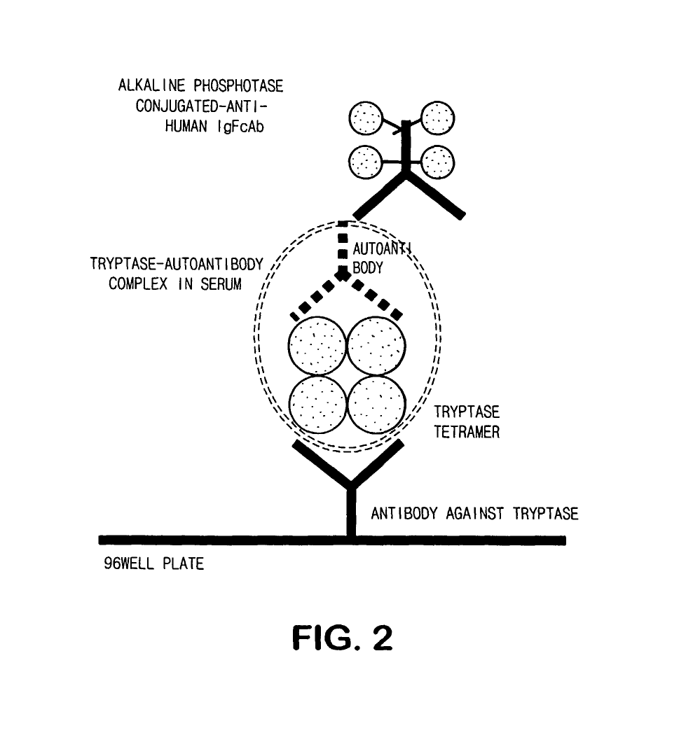

Anaphylatoxins, C3a and C5a, can enhance cutaneous vascular permeability and contract ileal smooth muscle of guinea pigs, directly initiate histamine release from rat mast cells, and cause wheal and flare formation when injected into human skin (16-20). Thus anaphylatoxic peptides, C3a and C5a, can trigger a variety of responses that play an important role in generating anaphylaxis, allergic and inflammatory reactions. Complement 3 (C3) plays a central role in the activation of the complement system. C3 is composed of two subunits, α-chain with 120 kDa M.W. and β-chain with 75 kDa M.W. Physiologic activation of C3 to C3b (185 kDa) and C3a (9 kDa) occurs by a selective proteolytic cleavage of the α-chain by either the classical C3 convertase (C3b,Bb) or the alternative C3 convertase (C4b,2a). C3b can bind to antigen through a thio ester bond and enhance Ag internalization and presentation to Ag-specific T lymphocytes (21). This suggests that after proteolytic activation in an area of inflammation, C3 may play a role in delivering Ag to APCs and give rise to enhance humoral immune response. There is only one publication describing that human mast cell tryptase stabilized by heparin could cleave C3 to yield C3a in vitro in a reaction modulated by heparin at neutral pH, but did not appear to affect anaphylatoxin since the reaction exhibited very limited capacity to generate C3a anaphylatoxin from purified human C3 (22). However, at acidic pH, tryptase may generate many biologicaly active products in vitro (23), such as anticoagulant fragment D and bradykinin (24). Thus, roles of human mast cell tryptase in complement and anaphylatoxin metabolism need to be reevaluated. Moreover, In vitro, studies show that antitryptase antibodies may enhance tryptase activity at acidic pH (24). Given the new insights into the effects of pH on tryptase proteolytic activity and existence of anti-mast cell tryptase autoantibodies depending our preliminary results, we propose that the released tryptase after mast cell activation does not generate active C3a and C5a without modulation by autoantibodies but degrades C3a and C5a (FIG. 1). When antitryptase autoantibodies are present, tryptase acts to generate the anaphylatoxins without subsequent degradation and inhibit degradation of the anaphylatoxins leading to anaphylactic events (FIG. 1). Thus, if autoantibody modulation can be blocked by using information of autoantibody binding epitope, the disease should be prevented.

For application of antitryptase-autoantibody complex as diagnostic marker and therapeutic target for immediate hypersensitivity diseases, there are three key points in the invention:

1. The clinical and pathological significance between the methods herein and U.S. Pat. No. 5,861,264 (to Kyle C. Elrod) are completely different. The disclosure herein provides methods of detecting antitryptase-autoantibody complex in patient sera with immediate hypersensitivity diseases, while U.S. Pat. No. 5,861,264 to Elrod describes anti-tryptase detection in inflammatory diseases, which are autoimmune diseases characterized by different autoantibodies that lack specificity. Thus, as shown herein, the complex is the cause of the diseases and free autoantibody is the effect of the diseases. For example, SLR contains a lot of autoantibodies against different self-proteins in nuclear, cytosol, cell membrane, and mast cell proteins as they claimed in the patent; it does not teach detection of autoantibody against mast cell tryptase. The assay herein permits diagnosis of immediate hypersensitivity diseases; U.S. Pat. No. 5,861,264 to Elrod may be relevant for inflammatory diseases.

2. Therapeutic significance is a unique advantage for our invention. Because of antitryptase-autoantibody complex is a cause of the diseases, the complex could serve as therapeutic target potentially, while the autoantibodies detected in U.S. Pat. No. 5,861,264 to Elrod do not have therapeutic significance. It detects autoantibody against mast cell tryptase, chymase, and other mast cell proteins, which is not a specific assay and the detected autoantibody is not related to disease occurrence and progress, and cannot be used as a therapeutic target.

3. The complex formed in the instant methods is different from that formed in the method of U.S. Pat. No. 5,861,264 to Elrod, and thus has different significance. U.S. Pat. No. 5,861,264 to Elrod provides a method by forming an immune complex of anti-tryptase autoantibody and tryptase in in vitro in a 96 well plate. In the methods provided herein, the presence of antitryptase autoantibody-tryptase immune complex in sera of patients with immediate hypersensitivity diseases is detected. This complex causes and progress the diseases. Therefore, the immune complex can used as therapeutic target.

Hence, the present invention relates to a method for diagnosing the diseases by using autoantibody-mast cell tryptase immune complex and the kit thereof. More specifically the present invention includes the discovery of autoantibody-mast cell tryptase immune complex in anaphylaxis serum and establishment of enzyme linked immunosorbent assays (ELISA) for detection of autoantibody-mast cell tryptase immune complex in patient sera with anaphylaxis.

### EXAMPLE 1

The kit for Human Mast Cell Tryptase-Autoantibody Complex ELISA includes:

1. Coating Buffer(0.01 M Tris, 0.12 g, 0.15 M NaCl, 0.88 g, 0.05%, NaN3, 0.1 g, pH 8.5, for 100 ml)

2. Washing Buffer(0.01 M HEPES, 2.38 g, 0.5 M NaCl, 29.2 g, 25 mM EDTA, 9.21 g, 0.1% BSA 1.0 g, 0.05% NaN3, 0.5 g, 0.05% Tween 20, 0.5 ml. Add H2O up to 1000 ml, pH 7.4)

3. Blocking Buffer(0.01 M HEPES, 2.38 g, 0.5 M NaCl, 29.2 g, 25 mM EDTA, 9.21 g, 3% BSA, 30 g, 0.05% NaN3, 0.5 g. Add H2O up to 1000 ml, pH 7.4);

4. Binding Buffer(0.01 M HEPES, 2.38 g, 0.15 M NaCl, 29.2 g, 25 mM EDTA, 9.21 g, 0.1% BSA 1.0 g, 0.05% NaN3, 0.5 g. Add H2O up to 1000 ml, pH 7.4);

5. DEA Buffer(Diethanolamine, 48.5 ml, Magnesium Chloride, 0.05 g, 0.1% NaN3, 0.1 g, pH 9.8, for 500 ml;

6. Capture mAb against human mast cell tryptase; Detector, alkaline phosphotase conjugated mAb against human IgG Fc fragments; and

7. Human lung mast cell tryptase.

### EXAMPLE 2

Method for detecting Human Mast Cell Tryptase-Autoantibody Complex ELISA

1. Coat 96 well PRO-BIND plates with capture mAb for 1.5 hrs at room temperature with 100 ul of 2.5 ug/ml in Coating Buffer.

2. Block with 200 ul of Blocking Buffer 1 hr at room temperature.

3. Wash with dH₂O and slap it out.

4. Add 100 ul of Binding Buffer and 10 ul of 1:100 diluted normal or patient sera for tryptase assay.

5. Incubate at room temperature for 1 hr. or overnight at 4° C.

6. Wash plates with Washing buffer 5 times and dH₂O once. Slap out plates.

7. Add 100 ul of 1:10,000 diluted Alkaline Phosphotase conjugated antibody and incubate at room temperature for 1 hr.

8. Wash with Washing Buffer 5 times, dH₂O once and slap out.

9. Develop with 100 ul/well of p-Nitrophenyl Phosphate solution (1 tablet in 15 ml DEA buffer).

10. Incubation at 37° C., O.D at 405 nm ELISA Reader at 30 min and 1 hr. Result & conclusion:

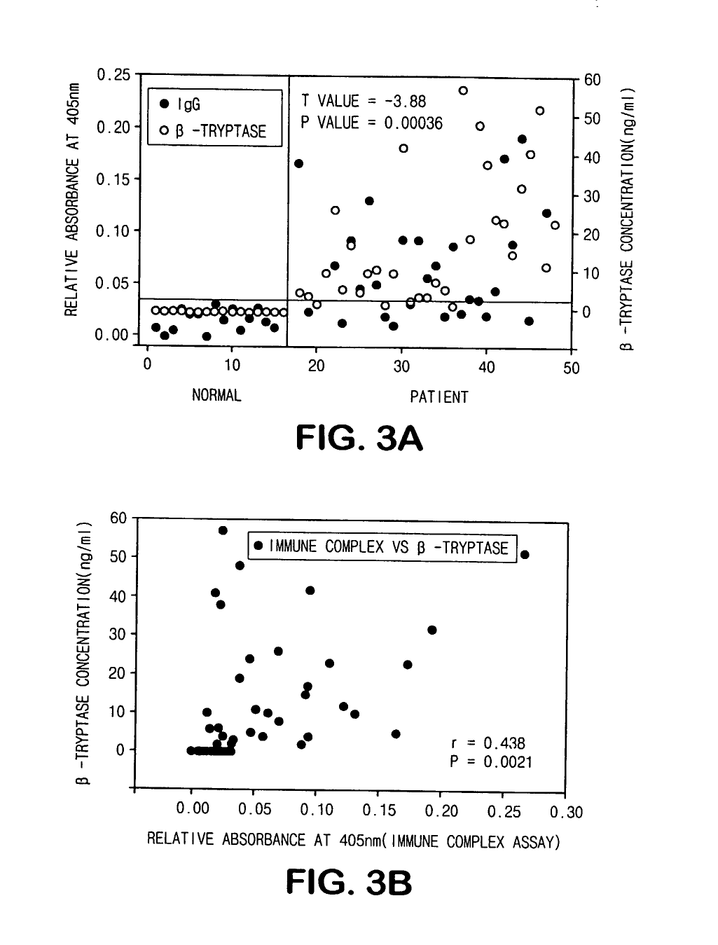

We have optimized the double antibody sandwich ELISA assay and detected the presence of high titers (1:1,000) of the immune complex in twenty one of thirty of the patient sera with high β-tryptase levels, an unique marker for systematic anaphylaxis (FIG. 3A). Among them high titers of the immune complex were detected in six of nine sera of postmortem sera with high β-tryptase. The difference of the titers between survival patient and postmortem is not significance. Pearson Correlation Coefficiency analysis shows that the correlation between β-tryptase concentration and the titers of antitryptase autoantibody-tryptase immune complex is significant (P=0.0021, r2=0.438) (FIG. 3B). All of the patient samples were suspected to have had anaphylactic reaction and sent for tryptase level assays in Allergy and Immunology Laboratory.

However, not all of sera with high β-tryptase levels show high titers of immune complex suggesting that not all of patients with high β-tryptase may have anaphylactic reaction and not all of postmortem with high β-tryptase was caused by anaphylaxis, which is consistent with the previous report (84). It is also possible that anaphylaxis might be caused by more than one mechanism.

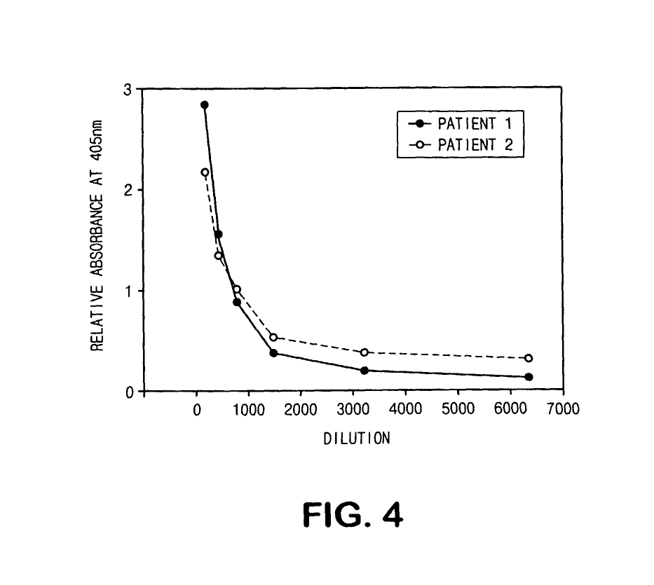

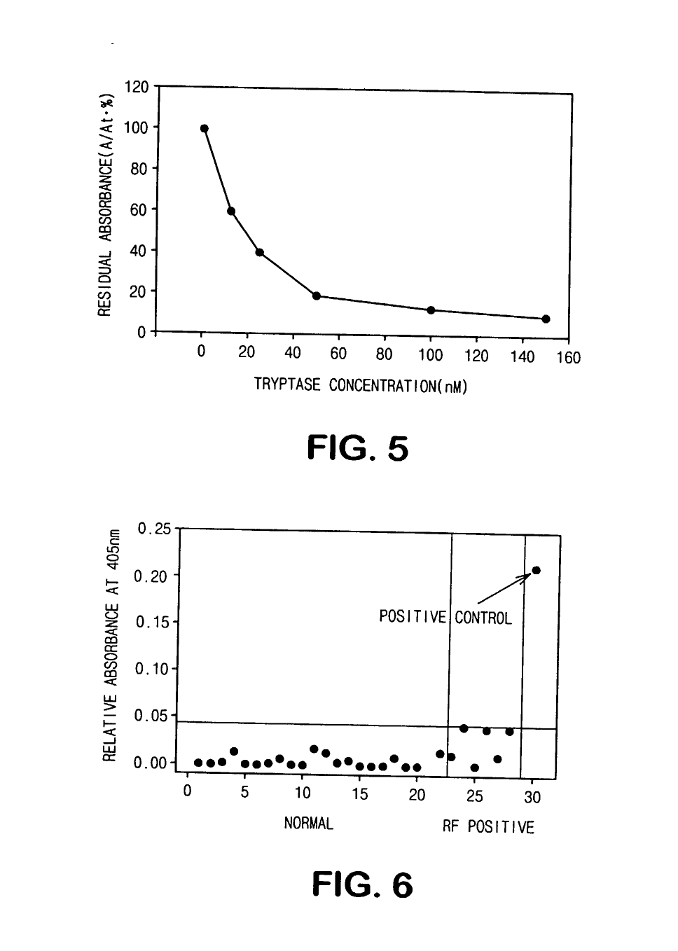

To address the specificity of ELISA assay measurement, we did dose and competitive experiment. FIG. 4 shows the dose response. As increase dilution of sera with binding buffer, the relative absorbance decreases. The results suggest that the binding is dose dependent. FIG. 5 shows the immune complex binding is competitive with purified human mast cell tryptase. The purified human lung mast cell tryptase was added to compete with the autoantibody-tryptase immune complex in patient serum for binding with the anti-tryptase antibody coated in 96-well plate. As we increased concentration of the purified tryptase in the wells, the detectable absorbance of autoantibody-tryptase immune complex decreased suggesting that the binding with the immune complex is competitive with exogenously adding tryptase and is specific to human mast cell tryptase (FIG. 5).

Eight patient sera were used for competitive experiments. Adding the purified human mast cell tryptase suggesting the presence of free anti-tryptase autoantibody in the patient sera, which is absent in normal subjects, cannot be inhibited about 10-30% of the maximum detectable absorbance of autoantibody-tryptase immune complex.

Rheumatoid factors (RF) can interfere with antigen capture type assays, we have tested effects of RF on our ELISA assay using RF (0 to 333 units per ml)-containing sera provided by Dr. George Moxley, Rheumatitis Research Laboratory, Medical College of Virginia, Richmond, Va. The negative control sera are from patients with no RF and positive controls from patient with immediate hypersensitivity diseases, high level of β-tryptase, and high titer of anti-tryptase autoantibody-tryptase immune complex. Three of six samples have very weak positive just above upper normal range (FIG. 6). This positive value has no relationship with titers of RF units suggesting the positive is not caused by the presence of rheumatoid factor.

The foregoing invention has been described in some detail by way of illustration and example, for purposes of clarity and understanding. It will be apparent to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

Reference List

1. Andrejevic,S., Bogic,M., Bukilica,M., Jovicic,Z., and Bonaci-Nikolic,B. 1998. [Laboratory diagnosis of allergic inflammation]. Srp.Arh.Celok.Lek. 126:499-505.

2. Ansari,M. Q., Zamora,J. L., and Lipscomb,M. F. 1993. Postmortem diagnosis of acute anaphylaxis by serum tryptase analysis. A case report. Am.J.Clin.Pathol. 99:101-103.

3. Benson,M. D. and Lindberg,R. E. 1996. Amniotic fluid embolism, anaphylaxis, and tryptase. Am.J.Obstet.Gynecol. 175:737.

4. Berger,P., Compton,S. J., Molimard,M., Walls,A. F., N'Guyen,C., Marthan,R., and Tunon-De-Lara,J. M. 1999. Mast cell tryptase as a mediator of hyperresponsiveness in human isolated bronchi. Clin.Exp.Allergy 29:804-812.

5. Bingham, C. O., III and Austen,K. F. 2000. Mast-cell responses in the development of asthma. J.Allergy Clin.Immunol. 105:S527-S534.

6. Brockow, K., Vieluf, D., Puschel, K., Grosch, J., and Ring, J. 1999. Increased postmortem serum mast cell tryptase in a fatal anaphylactoid reaction to nonionic radiocontrast medium. J.Allergy Clin.Immunol. 104:237-238.

7. Butrus, S. I., Negvesky, G. J., Rivera-Velazques,P. M., and Schwartz,L. B. 1999. Serum tryptase: an indicator of anaphylaxis following fluorescein angiography. Graefes Arch.Clin.Exp.Ophthalmol. 237:433-434.

8. Castells, M. and Schwartz, L. B. 1988. Tryptase levels in nasal-lavage fluid as an indicator of the immediate allergic response. J.Allergy Clin.Immunol. 82:348-355.

9. Eberlein-Konig, B., Ullmann, S., Thomas, P., and Przybilla, B. 1995. Tryptase and histamine release due to a sting challenge in bee venom allergic patients treated successfully or unsuccessfully with hyposensitization. Clin.Exp.Allergy 25:704-712.

10. Edston, E., Hage-Hamsten, M., and Johansson, S. G. 1996. Tryptase—at last a useful diagnostic marker for anaphylactic death. Allergy 51:443-445.

11. Edston, E. and Hage-Hamsten, M. 1997. Anaphylactoid shock—a common cause of death in heroin addicts? Allergy 52:950-954.

12. Edston, E. and Hage-Hamsten, M. 1998. beta-Tryptase measurements post-mortem in anaphylactic deaths and in controls. Forensic Sci.Int. 93:135-142.

13. Edston,E., Gidlund,E., Wickman,M., Ribbing,H., and Hage-Hamsten,M. 1999. Increased mast cell tryptase in sudden infant de. Clin.Exp.Allergy 29:1648-1654.

14. Enander,I., Matsson,P., Nystrand,J., Andersson,A. S., Eklund,E., Bradford,T. R., and Schwartz,L. B. 1991. A new radioimmunoassay for human mast cell tryptase using monoclonal antibodies. J.Immunol.Methods 138:39-46.

15. Enrique,E., Garcia-Ortega,P., Sotorra,O., Gaig,P., and Richart,C. 1999. Usefulness of UniCAP-Tryptase fluoroimmunoassay in the diagnosis of anaphylaxis. Allergy 54:602-606.

16. Fineschi,V., Monasterolo,G., Rosi,R., and Turillazzi,E. 1999. Fatal anaphylactic shock during a fluorescein angiography. Forensic Sci.Int. 100:137-142.

17. Fisher,M. 1991. Tryptase in anaphylaxis [letter]. Anaesth.Intensive Care 19:479.

18. Fisher,M. M. and Baldo,B. A. 1993. The diagnosis of fatal anaphylactic reactions during anaesthesia: employment of immunoassays for mast cell tryptase and drug-reactive IgE antibodies. Anaesth.Intensive Care 21:353-357.

19. Fisher,M. M. and Baldo,B. A. 2000. Immunoassays in the diagnosis of anaphylaxis to neuromuscular blocking drugs: the value of morphine for the detection of IgE antibodies in allergic subjects. Anaesth.Intensive Care 28:167-170.

20. Hagan,L. L., Goetz,D. W., Revercomb,C. H., and Garriott,J. 1998. Sudden infant death syndrome: a search for allergen hypersensitivity. Ann.Allergy Asthma Immunol. 80:227-231.

21. Hamilton,R. G. 1997. Laboratory analyses in the diagnosis of human allergic disease. Methods 13:25-32.

22. Jarvikallio,A., Naukkarinen,A., Harvima,I. T., Aalto,M. L., and Horsmanheimo,M. 1997. Quantitative analysis of tryp. Br.J.Dermatol. 136:871-877.

23. Katunuma,N. and Kido,H. 1988. Biological functions of serine proteases in mast cells in allergic inflammation. J.Cell Biochem. 38:291-301.

24. Laroche,D., Vergnaud,M. C., Sillard,B., Soufarapis,H., and Bricard,H. 1991. Biochemical markers of anaphylactoid reactions to drugs. Comparison of plasma histamine and tryptase. Anesthesiology 75:945-949.

25. Laroche,D., Dubois,F., Lefrancois,C., Vergnaud,M. C., Gerard,J. L., Soufarapis,H., Sillard,B., and Bricard,H. 1992. [Early biological markers of anaphylactoid reactions occurring during anesthesia]. Ann.Fr.Anesth.Reanim. 11:613-618.

26. Laroche,D., Namour,F., Lefrancois,C., Aimone-Gastin,I., Romano, A., Sainte-Laudy, J., Laxenaire,M. C., and Gueant,J. L. 1999. Anaphylactoid and anaphylactic reactions to iodinated contrast material. Allergy 54 Suppl 58:13-16.

27. Lin,R. Y., Schwartz,L. B., Curry,A., Pesola,G. R., Knight,R. J., Lee,H. S., Bakalchuk,L., Tenenbaum,C., and Westfal,R. E. 2000. Histamine and tryptase levels in patients with acute allergic reactions: An emergency department-based study. J.Allergy Clin.Immunol. 106:65-71.

28. Ludolph-Hauser, D., Rueff, F., Sommerhoff, C. P., and Przybilla, B. 1999. [Tryptase, a marker for the activation and localization of mast cells]. Hautarzt 50:556-561.

29. Lynch, J. P., Renz,C. L., Laroche,D., and Moss,J. 2000. Value of baseline levels in assessing tryptase release. Inflamm.Res. 49 Suppl 1:S23-S24.

30. Martinez, I. E., Rojas,R. E., and Orea,S. M. 1996. [Tryptase: a diagnostic marker in allergic diseases]. Rev.Alerg.Mex. 43:73-76.

31. Neugut, A. I., Ghatak,A. T., and Miller,R. L. 2001. Anaphylaxis in the United States: An Investigation Into Its Epidemiology. Arch.Intern.Med. 161:15-21.

32. Ordoqui, E., Zubeldia,J. M., Aranzabal,A., Rubio,M., Herrero,T., Tornero,P., Rodriguez,V. M., Prieto,A., and Baeza,M. L. 1997. Serum tryptase levels in adverse drug reactions. Allergy 52:1102-1105.

33. Ownby, D. R., Tomlanovich,M., Sammons,N., and McCullough,J. 1991. Anaphylaxis associated with latex allergy during barium enema examinations. AJR Am.J.Roentgenol. 156:903-908.

34. Platt, M. S., Yunginger,J. W., Sekula-Perlman,A., Irani,A. M., Smialek,J., Mirchandani,H. G., and Schwartz,L. B. 1994. Involvement of mast cells in sudden infant death syndrome. J.Allergy Clin.Immunol. 94:250-256.

35. Prahlow, J. A. and Barnard,J. J. 1998. Fatal anaphylaxis due to fire ant stings. Am.J.Forensic Med.Pathol. 19:137-142.

36. Proud, D., Bailey, G. S., Naclerio, R. M., Reynolds, C. J., Cruz, A. A., Eggleston, P. A., Lichtenstein, L. M., and Togias, A. G. 1992. Tryptase and histamine as markers to evaluate mast cell activation during the responses to nasal challenge with allergen, cold, dry air, and hyperosmolar solutions. J.Allergy Clin.Immunol. 89:1098-1110.

37. Ren, S., Lawson, A. E., Carr, M., Baumgarten, C. M., and Schwartz,L. B. 1997. Human tryptase fibrinogenolysis is optimal at acidic pH and generates anticoagulant fragments in the presence of the anti-tryptase monoclonal antibody B12. J.Immunol. 159:3540-3548.

38. Ren, S., Sakai, K., and Schwartz, L. B. 1998. Regulation of human mast cell beta-tryptase: conversion of inactive monomer to active tetramer at acid pH. J.Immunol. 160:4561-4569.

39. Rice, K. D., Tanaka, R. D., Katz, B. A., Numerof, R. P., and Moore, W. R. 1998. Inhibitors of tryptase for the treatment of mast cell-mediated diseases. Curr.Pharm.Des 4:381-396.

40. Rice, K. D., Wang, V. R., Gangloff, A. R., Kuo, E. Y., Dener, J. M., Newcomb, W. S., Young, W. B., Putnam, D., Cregar,L., Wong, M. et al. 2000. Dibasic inhibitors of human mast cell tryptase. Part 2: structure-activity relationships and requirements for potent activity [In Process Citation]. Bioorg. Med.Chem.Lett. 10:2361-2366.

41. Rice, K. D., Gangloff, A. R., Kuo, E. Y., Dener, J. M., Wang, V. R., Lum, R., Newcomb, W. S., Havel, C., Putnam, D., Cregar, L. et al. 2000. Dibasic inhibitors of human mast cell tryptase. Part 1: synthesis and optimization of a novel class of inhibitors [In Process Citation]. Bioorg.Med.Chem.Lett. 10:2357-2360.

42. Schwartz, H. J., Yunginger, J. W., and Schwartz, L. B. 1995. Is unrecognized anaphylaxis a cause of sudden unexpected death? Clin.Exp.Allergy 25:866-870.

43. Schwartz, L. B., Lewis, R. A., and Austen, K. F. 1981. Tryptase from human pulmonary mast cells. Purification and characterization. J.Biol.Chem. 256:11939-11943.

44. Schwartz, L. B., Atkins, P. C., Bradford, T. R., Fleekop, P., Shalit,M., and Zweiman, B. 1987. Release of tryptase together with histamine during the immediate cutaneous response to allergen. J.Allergy Clin.Immunol. 80:850-855.

45. Schwartz, L. B., Metcalfe, D. D., Miller, J. S., Earl, H., and Sullivan, T. 1987. Tryptase levels as an indicator of mast-cell activation in systemic anaphylaxis and mastocytosis. N.Engl.J.Med. 316:1622-1626.

46. Schwartz, L. B. 1994. Tryptase: a clinical indicator of mast cell-dependent events. Allergy Proc. 15:119-123.

47. Tanaka, R. D., Clark, J. M., Warne, R. L., Abraham, W. M., and Moore,W. R. 1995. Mast cell tryptase: a new target for therapeutic intervention in asthma. Int.Arch.Allergy Immunol. 107:408-409.

48. Tanus, T., Mines, D., Atkins, P. C., and Levinson, A. I. 1994. Serum tryptase in idiopathic anaphylaxis: a case report and review of the literature. Ann.Emerg.Med. 24:104-107.

49. Watkins, J. and Wild, G. 1993. Improved diagnosis of anaphylactoid reactions by measurement of serum tryptase and urinary methylhistamine. Ann.Fr.Anesth.Reanim. 12:169-172.

50. Galli, S. J., Maurer, M., and Lantz, C. S. 1999. Mast cells as sentinels of innate immunity. Curr.Opin.Immunol. 11:53-59.

51. Schwartz, L. B. 1990. Tryptase from human mast cells: biochemistry, biology and clinical utility. Monogr Allergy 27:90-113.

52, Miller, J. S., Westin, E. H., and Schwartz, L. B. 1989. Cloning and characterization of complementary DNA for human tryptase. J.Clin.Invest 84:1188-1195.

53. Miller, J. S., Moxley, G., and Schwartz, L. B. 1990. Cloning and characterization of a second complementary DNA for human tryptase. J.Clin.Invest 86:864-870.

54. Vanderslice, P., Craik, C. S., Nadel, J. A., and Caughey, G. H. 1989. Molecular cloning of dog mast cell tryptase and a related protease: structural evidence of a unique mode of serine protease activation. Biochemistry 28:4148-4155.

55. Schwartz, L. B., Sakai, K., Bradford, T. R., Ren, S., Zweiman, B., Worobec, A. S., and Metcalfe, D. D. 1995. The alpha form of human tryptase is the predominant type present in blood at baseline in normal subjects and is elevated in those with systemic mastocytosis. J.Clin.Invest 96:2702-2710.

56. Alter, S. C. and Schwartz, L. B. 1989. Effect of histamine and divalent cations on the activity and stability of tryptase from human mast cells. Biochim.Biophys.Acta 991:426-430.

57. Alter, S. C., Metcalfe, D. D., Bradford, T. R., and Schwartz, L. B. 1987. Regulation of human mast cell tryptase. Effects of enzyme concentration, ionic strength and the structure and negative charge density of polysaccharides. Biochem.J. 248:821-827.

58. Sakai, K., Ren, S., and Schwartz, L. B. 1996. A novel heparin-dependent processing pathway for human tryptase. Autocatalysis followed by activation with dipeptidyl peptidase I [see comments]. J.Clin.Invest 97:988-995.

59. Burgess, L. E., Newhouse, B. J., Ibrahim, P., Rizzi, J., Kashem, M. A., Hartman, A., Brandhuber, B. J., Wright, C. D., Thomson, D. S., Vigers, G. P. et al. 1999. Potent selective nonpeptidic inhibitors of human lung tryptase. Proc.Natl.Acad.Sci.U.S.A 96:8348-8352.

60. Pereira, P. J., Bergner, A., Macedo-Ribeiro, S., Huber, R., Matschiner, G., Fritz, H., Sommerhoff, C. P., and Bode, W. 1998. Human beta-tryptase is a ring-like tetramer with active sites facing a central pore. Nature 392:306-311.

61. Alter, S. C., Kramps,J. A., Janoff,A., and Schwartz,L. B. 1990. Interactions of human mast cell tryptase with biological protease inhibitors. Arch.Biochem.Biophys. 276:26-31.

62. Schwartz, L. B., Bradford,T. R., Littman,B. H., and Wintroub,B. U. 1985. The fibrinogenolytic activity of purified tryptase from human lung mast cells. J.Immunol. 135:2762-2767.

63. Gruber, B. L., Schwartz,L. B., Ramamurthy,N. S., Irani,A. M., and Marchese,M. J. 1988. Activation of latent rheumatoid synovial collagenase by human mast cell tryptase. J.Immunol. 140:3936-3942.

64. Lohi, J., Harvima, I., and Keski-Oja,J. 1992. Pericellular substrates of human mast cell tryptase: 72,000 dalton gelatinase and fibronectin. J.Cell Biochem. 50:337-349.

65. Tam, E. K. and Caughey,G. H. 1990. Degradation of airway neuropeptides by human lung tryptase. Am.J.Respir.Cell Mol.Biol. 3:27-32.

66. Stack,M. S. and Johnson,D. A. 1994. Human mast cell tryptase activates single-chain urinary-type plasminogen activator (pro-urokinase). J.Biol.Chem. 269:9416-9419.

67. Sekizawa,K., Caughey,G. H., Lazarus,S. C., Gold,W. M., and Nadel,J. A. 1989. Mast cell tryptase causes airway smooth muscle hyperresponsiveness in dogs. J.Clin.Invest 83:175-179.

68. Hartmann,T., Ruoss,S. J., Raymond,W. W., Seuwen,K., and Caughey,G. H. 1992. Human tryptase as a potent, cell-specific mitogen: role of signaling pathways in synergistic responses. Am.J.Physiol 262:L528-L534.

69. Cairns,J. A. and Walls,A. F. 1996. Mast cell tryptase is a mitogen for epithelial cells. Stimulation of IL-8 production and intercellular adhesion molecule-1 expression. J.Immunol. 156:275-283.

70. Brown,J. K., Tyler,C. L., Jones,C. A., Ruoss,S. J., Hartmann,T., and Caughey,G. H. 1995. Tryptase, the dominant secretory granular protein in human mast cells, is a potent mitogen for cultured dog tracheal smooth muscle cells. Am.J.Respir.Cell Mol.Biol. 13:227-236.

71. Brown,J. K., Jones,C. A., Tyler,C. L., Ruoss,S. J., Hartmann,T., and Caughey,G. H. 1995. Tryptase-induced mitogenesis in airway smooth muscle cells. Potency, mechanisms, and interactions with other mast cell mediators. Chest 107:95S-96S.

72. Cairns,J. A. 1998. Mast cell tryptase and its role in tissue remodelling [editorial; comment]. Clin.Exp.Allergy 28:1460-1463.

73. Walls,A. F., He,S., Teran,L. M., Buckley,M. G., Jung,K. S., Holgate,S. T., Shute,J. K., and Cairns,J. A. 1995. Granulocyte recruitment by human mast cell tryptase. Int.Arch.Allergy Immunol. 107:372-373.

74. Proud,D., Siekierski,E. S., and Bailey,G. S. 1988. Identification of human lung mast cell kininogenase as tryptase and relevance of tryptase kininogenase activity. Biochem.Pharmacol. 37:1473-1480.

75. Schwartz,L. B., Bradford,T. R., Rouse,C., Irani,A. M., Rasp,G., Van der Zwan,J. K., and Van der Linden,P. W. 1994. Development of a new, more sensitive immunoassay for human tryptase: use in systemic anaphylaxis. J.Clin.Immunol. 14:190-204.

76. Johnson,P. R., Ammit,A. J., Carlin,S. M., Armour,C. L., Caughey,G. H., and Black,J. L. 1997. Mast cell tryptase potentiates histamine-induced contraction in human sensitized bronchus. Eur.Respir.J. 10:38-43.

77. Barrios,V. E., Middleton,S. C., Kashem,M. A., Havill,A. M., Toombs,C. F., and Wright,C. D. 1998. Tryptase mediates hyperresponsiveness in isolated guinea pig bronchi. Life Sci. 63:2295-2303.

78. Clark,J. M., Abraham,W. M., Fishman,C. E., Forteza,R., Ahmed,A., Cortes,A., Warne,R. L., Moore,W. R., and Tanaka,R. D. 1995. Tryptase inhibitors block allergen-induced airway and inflammatory responses in allergic sheep. Am.J.Respir.Crit Care Med. 152:2076-2083.

79. Molinari,J. F., Moore,W. R., Clark,J., Tanaka,R., Butterfield,J. H., and Abraham,W. M. 1995. Role of tryptase in immediate cutaneous responses in allergic sheep. J.Appl.Physiol 79:1966-1970.

80. Molinari,J. F., Scuri,M., Moore,W. R., Clark,J., Tanaka,R., and Abraham,W. M. 1996. Inhaled tryptase causes bronchoconstriction in sheep via histamine release. Am.J.Respir.Crit Care Med. 154:649-653.

81. De Sanctis,G. T., Merchant,M., Beier,D. R., Dredge,R. D., Grobholz,J. K., Martin,T. R., Lander,E. S., and Drazen,J. M. 1995. Quantitative locus analysis of airway hyperresponsiveness in A/J and C57BL/6J mice. Nat.Genet. 11:150-154.

82. Ghildyal,N., Friend,D. S., Freelund,R., Austen,K. F., McNeil,H. P., Schiller,V., and Stevens,R. L. 1994. Lack of expression of the tryptase mouse mast cell protease 7 in mast cells of the C57BL/6J mouse. J.Immunol. 153:2624-2630.

83. Hunt,J. E., Stevens,R. L., Austen,K. F., Zhang,J., Xia,Z., and Ghildyal,N. 1996. Natural disruption of the mouse mast cell protease 7 gene in the C57BL/6 mouse. J.Biol.Chem. 271:2851-2855.

84. Franconi,G. M., Graf,P. D., Lazarus,S. C., Nadel,J. A., and Caughey,G. H. 1989. Mast cell tryptase and chymase reverse airway smooth muscle relaxation induced by vasoactive intestinal peptide in the ferret. J.Pharmacol.Exp.Ther. 248:947-951.

85. Ollerenshaw,S., Jarvis,D., Woolcock,A., Sullivan,C., and Scheibner,T. 1989. Absence of immunoreactive vasoactive intestinal polypeptide in tissue from the lungs of patients with asthma [see comments]. N.Engl.J.Med. 320:1244-1248.

86. Lilly,C. M., Drazen,J. M., and Shore,S. A. 1993. Peptidase modulation of airway effects of neuropeptides. Proc.Soc.Exp.Biol.Med. 203:388-404.

87. Lilly,C. M., Martins,M. A., and Drazen,J. M. 1993. Peptidase modulation of vasoactive intestinal peptide pulmonary relaxation in tracheal superfused guinea pig lungs. J.Clin.Invest 91:235-243.

88. Lilly,C. M., Kobzik,L., Hall,A. E., and Drazen,J. M. 1994. Effects of chronic airway inflammation on the activity and enzymatic inactivation of neuropeptides in guinea pig lungs. J.Clin.Invest 93:2667-2674.

89. Gerard,C. and Hugli,T. E. 1981. C5a: a mediator of chemotaxis and cellular release reactions. Kroc.Found.Ser. 14:147-160.

90. Hugli,T. E. 1986. Biochemistry and biology of anaphylatoxins [published erratum appears in Complement 1987;4(2):following 119]. Complement 3:111-127.

91. Johnson,R. J. and Chenoweth,D. E. 1985. Structure and function of human C5a anaphylatoxin. Selective modification of tyrosine 23 alters biological activity but not antigenicity. J.Biol.Chem. 260:10339-10345.

92. Lambris,J. D., Reid,K. B., and Volanakis,J. E. 1999. The evolution, structure, biology and pathophysiology of complement. Immunol.Today 20:207-211.

93. Muller-Eberhard,H. J. The anaphylatoxins: formation, structure, function and control. pp. 339-52. In: Johansson.S G, et al., ed.Molecular.and biological.aspects of the.acute.allergic.reaction.New York., Plenum.Press, 1976.W3.NO369.1976m.

94. Morgan,E. L., Thoman,M. L., Hoeprich,P. D., and Hugli,T. E. 1985. Bioactive complement fragments in immunoregulation. Immunol.Lett. 9:207-213.

95. Schwartz,L. B., Kawahara,M. S., Hugli,T. E., Vik,D., Fearon,D. T., and Austen,K. F. 1983. Generation of C3a anaphylatoxin from human C3 by human mast cell tryptase. J.Immunol. 130:1891-1895.

What is claimed is:

1. An immunoassay method for detecting human tryptase-autoantibody immune complex comprising:
coating a solid support with capture antibodies;
blocking the solid support;
adding a binding buffer and a serum sample on the solid support;
adding detection antibodies on the solid support; and
determining the presence of human tryptase-autoantibody complex.

2. The method of claim 1, further comprising washing steps.

3. The method of claim 1, wherein the method is the method for detecting an anaphylaxis disease.

4. The method of claim 1, wherein the method is a double antibody-sandwich enzyme-linked immunosorbent assay (ELISA).

5. The method of claim 1, wherein the capture antibody is the antibody against human mast cell tryptase.

6. The method of claim 1, wherein the detection antibody is an alkaline phosphotase conjugated monoclonal antibody against human IgG Fc fragment.

7. A kit for performing the detecting method of claim 1, said kit comprising:
the capture monoclonal antibody against human mast cell tryptase; and
the alkaline phosphotase conjugated monoclonal antibody against human IgG Fc fragment as a detection antibody.
